(19) United States
(12) Patent Application Publication
(10) Pub. No.: US 2017/0049604 A1
(43) Pub. Date: Feb. 23, 2017

(54) SLEEVE SLING

(71) Applicant: Dwight KEMP, Winchester, VA (US)

(72) Inventor: Dwight KEMP, Winchester, VA (US)

(21) Appl. No.: 15/221,092

(22) Filed: Jul. 27, 2016

Related U.S. Application Data
(60) Provisional application No. 62/197,408, filed on Jul. 27, 2015.

(51) Int. Cl. A61F 5/37

(52) U.S. Cl. CPC A61F 5/3738

(57) ABSTRACT

A sleeve-like support member can be used to enhance the comfort and wearability of a convention sling. A conventional sling includes a pouch adapted for receiving a user's arm to be immobilized. The sleeve-like support member is shaped to fit around a user's shoulder, and further is adapted to attach to the sling pouch by one or more straps secured to and extending away from the pouch. An axillary strap can be secured to a bottom portion of the sleeve-like support member to secure the sleeve-like support member around a user's upper arm. By securing to a user's shoulder, the sleeve-like support member removes pressure ordinarily applied by conventional slings to a user's neck, and provides an anchor point opposite the arm or shoulder to be immobilized.

### CROSS REFERENCE TO RELATED APPLICATIONS

This application claims the benefit of U.S. provisional application Ser. No. 62/197,408, filed Jul. 27, 2015, the contents of which are incorporated herein by reference.

### FIELD OF INVENTION

This application is directed generally to a surgical sprint, brace, or bandage. More particularly, the application is directed to an orthopedic bandage, namely, a sling.

### BACKGROUND

The sling is a fundamental component of treating many upper body injuries. As ubiquitous as the use of the sling are patients' complaints of discomfort caused by use of the sling. As shown below, there have been numerous innovations pertaining to slings.

For example, U.S. Pat. No. 3,554,194 to Johnson teaches an arm sling including upper and lower loop or strap portions joined together to form a generally figure-eight configuration. The upper loop includes a padding including a plastic foam material encased within a fabric stockinet. The padding is adapted to bear against the nape of the patient's neck. The lower loop includes a padding of plastic foam material encased within a fabric stockinet. A pair of fabric straps are provided on the lower loop padding, and threadedly receive the lower strap portion to retain the lower padding in adjustable position therealong. A buckle is provided for adjusting the length of both the upper and lower loop strap portions.

U.S. Pat. No. 4,716,895 to Marques et al. teaches a sling for support of the arm, which includes an elongated cloth body having a relatively wide central region and relatively narrow end regions, and further having an arm extending outwardly from adjacent the central region. The body is mounted on one shoulder and supports the opposite arm without the need for any separate arm-containing pouch or pocket. Hook and loop fasteners are provided to secure the ends of the body to each other, and to secure the arm to the body.

U.S. Pat. No. 4,733,658 to Ruthven, Jr. teaches a one-piece pad of a generally L-shape. The pad is formed with a head-receiving opening, and includes on each of its sides, a plurality of attachment apparatus. A two-part folding action forms a sling for either arm, wrist, or hand. The sling immobilizes the injured arm, wrist, or hand in an upwardly inclined position above the heart, and also provides an elbow support.

U.S. Pat. No. 5,334,132 to Burkhead teaches an arm sling that includes a forearm support section having a resealable opening in a bottom panel for permitting exercise, and flexion and extension of a patient's forearm without removing the sling harness from the patient. A splint pocket is also provided for housing a removable splint for supporting the patient's hand in a neutral position. An external pocket encloses a foam cushion for conforming engagement with the patient's waist thus providing an abduction effect.

United States Patent Application Publication Number 2006/0013976 to Leiss teaches a neck protector that is a cylindrical-shaped tube made of soft and thick fleece fabric. The dimensions are approximately 20″ long with a 5½″ circumference. The neck protector is for people who are wearing arm slings, typically after a broken arm or shoulder surgery. The weight of the arm puts a tremendous amount of pressure on the strap of the sling that has a tendency to rub and dig into the user's neck skin. This in turn can become painful and irritating to the user. The neck protector creates a soft cushion around the strap, thereby protecting the skin from harm. The neck protector easily slides over the strap portion of the arm sling. The user then adjusts the neck protector to a comfortable position around the back of the neck.

U.S. Pat. No. 7,563,236 to Kazmierczak et al. teaches a shoulder sling for the treatment of, for example, gleno-humeral dislocations and subluxations, capsular shifts, postoperative rotator cuff repair, postoperative Bankart procedures, and soft tissue strains and repairs. The sling includes a support pillow that abuts the wearer's torso, and a pouch that receives the wearer's arm. A plurality of straps suspend the pillow and pouch from the wearer's non-treatment trapezius. One of the straps prevents the pillow from shifting relative to the user, thus securely maintaining the wearer's arm at desired angles of abduction and external rotation. The pillow further includes an attached exercise grip and an indicator line that aids a physician in properly fitting the sling to the wearer.

United States Patent Application Publication Number 2011/0213282 to Cortese teaches an arm sling having broad supporting area on the shoulder of the injured side supports the weight of the upper and lower arm and allows mobility of the upper arm and elbow. The other shoulder from which the non-injured arm depends bears no weight of the supported arm.

Despite the purported advances in sling development and design, patients generally are able to tolerate wearing a sling only for a short time due to discomfort created by the downward pressure on the patient's neck or trapezius. For example, patients often complain of discomfort as the strap, even if modified by a pad, cuts or pulls on the person's neck. This is problematic as some patients are required to endure wearing a sling for 6 to 8 weeks or more. Thus, there remains a need for a stabilizing arm sling that comfortably secures an arm in the sling against the body.

### SUMMARY

The present invention solves the foregoing problems by providing a sleeve-like support member and sling that uses the shoulder opposite the arm to be stabilized for support rather than relying on the neck to support the arm to be immobilized.

One aspect of the invention is a sleeve-like support member attached to the shoulder opposite the arm or shoulder to be stabilized using the axilla. The support member can attach to the opposite shoulder by an underarm adjustable axillary strap. Adjustable straps upon the superior aspect of the support member can permit varied attachment of the sling strap to the support member. The strap of a standard sling can be attached to the support member, therefore increasing the tolerance and compliance of sling use. The application is useful to avoid applying pressure on the neck such as when there are traumatic events affecting the neck, such as fractures, burns, lacerations, infections, or recent surgical wounds.

The sling can be a single unit incorporating the pouch portion, which cradles the arm, with or without a pad to abduct the shoulder, which is common in post-operative rotator cuff repair. The sleeve-like support member may serve independently and function to connect to a standard sling device. The sleeve-like support member can be attached to any device that needs to be carried, such as a backpack, a tool belt, a vacuum or other household device, a hunters game-carrying device, a hunter's or soldier's weapon or munitions carrying device, etc.

The opposite arm remains free to move, while the sleeve-like support member serves as an anchor from above/superior to the shoulder. The strap portion of the sling pouch can be attached above or superiorly through an epaulette capture. The sleeve-like support member can be adjusted to the user by hook and loop closures, clasp, buckle, or other mechanical fastening means known to one of skill in the art, and will connect in various needed circumferences around the axilla and upper arm. A sling using a sleeve-like support member does not depend on or touch the neck. The sling likewise optionally but preferably does not depend on the torso for support.

A second aspect of the invention is a pouch adapted for receiving a user's arm to be immobilized; a sleeve-like support member adapted to attach the pouch to the user's shoulder opposite the arm or shoulder to be immobilized; one or more straps secured to and extending away from the pouch and secured to the support member; and an axillary strap secured to a bottom portion of the sleeve-like support member and positionable around the user's upper arm.

A third aspect of the invention is a sleeve-like support member adapted for use with a sling pouch, wherein the sleeve-like support member is shaped to cover a user's shoulder opposite an arm or shoulder of a user to be immobilized.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a front view of a user wearing on apparatus of the present invention;

FIG. 2 is a front view of a user wearing one of many alternative embodiments of an apparatus of the present invention;

FIG. 3 is a front view of a sleeve-like support member;

FIG. 4 is a front view of one of many possible alternative embodiments of a sleeve-like support member;

FIG. 5 is a front view of one of many possible alternative embodiments of a sleeve-like support member; and

FIG. 6 is a front view of an apparatus of the present invention.

### DETAILED DESCRIPTION

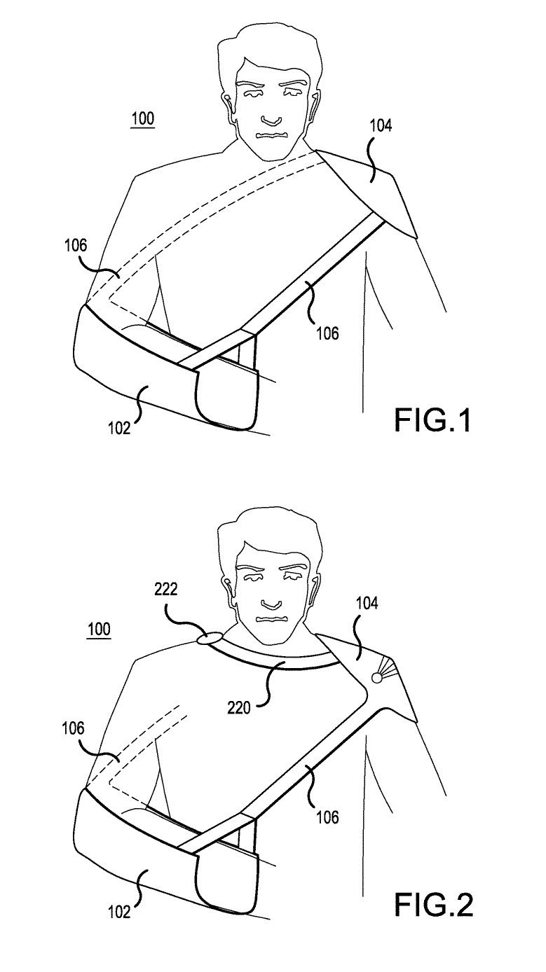

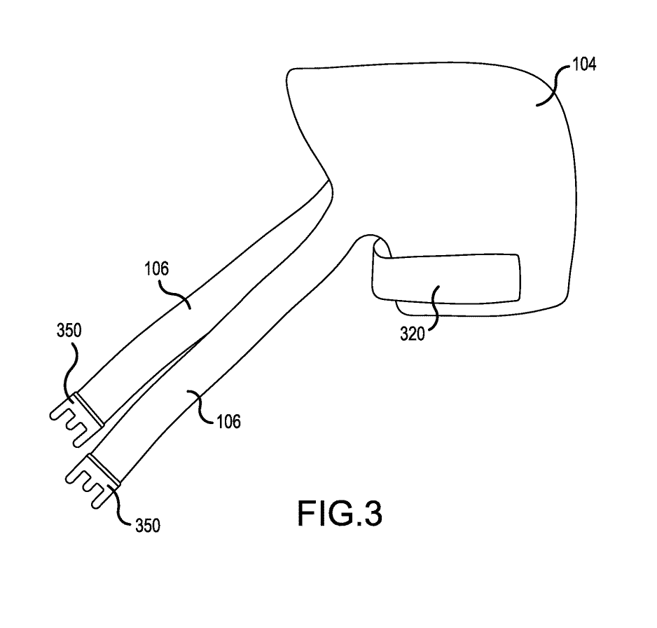

An apparatus 100 of the present invention is shown generally in FIG. 1. The apparatus 100 includes a pouch 102 (or “sling pouch”) adapted for receiving a user's arm to be immobilized. A sleeve-like support member 104 is attached to the pouch 102. The pouch 102 and sleeve-like support member 104 can be one contiguous piece or the sleeve-like support member 104 can be detachably connected to the pouch 102. The sleeve-like support member 104 is shaped to cover a user's shoulder opposite an arm or shoulder of a user to be immobilized. One or more straps 106 can be secured to and extend away from the pouch 102 to connect the pouch 102 and sleeve-like support member 104. An axillary strap 320 (FIG. 3) optionally but preferably is connected to a bottom portion of the sleeve-like support member 104 and is positionable around a user's upper arm.

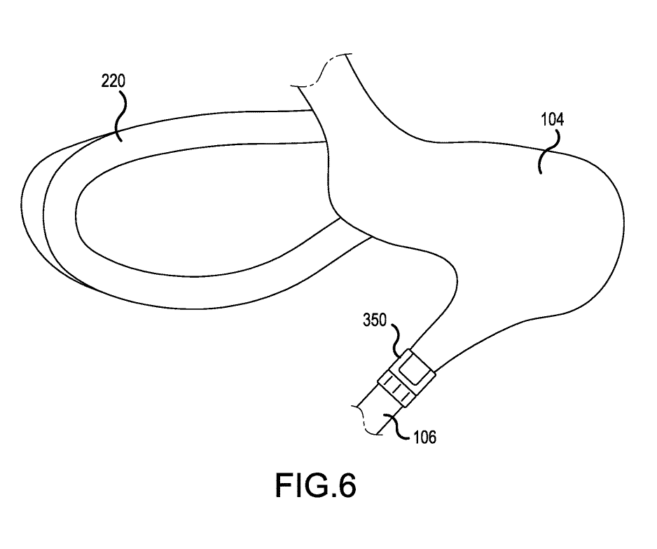

FIGS. 2 and 6 show alternative embodiments of the apparatus 100, which includes an optional neck strap 220 connected to a top portion of the sleeve-like support member 104. The neck strap 220 secures the sleeve-like support member 104 around the user's neck, and optionally but preferably includes a pad 222 positionable on the neck strap 220 to reduce pressure on the user's neck.

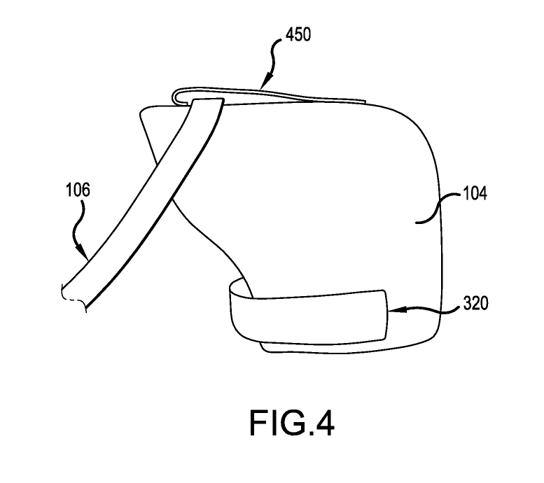

FIG. 3 shows a sleeve-like support member 104 that is detachably connectable to a pouch 102 of a conventional sling. Providing means for detachably connecting the sleeve-like member 104 to a conventional sling allows the sleeve-like member 104 to be retrofitted and used with conventional slings. An axillary strap 320 can be secured to a bottom portion of the sleeve-like support member 104. The axillary strap 320 can be positioned around a user's upper arm. The sleeve-like support member 104 can include one or more straps 106 for detachably connecting sleeve-like support member 104 to the pouch 102. Buckles 350 can be included at the ends of the one or more straps 106 for fastening the straps 106 to the pouch 102. Alternatively as shown in FIG. 4, the sleeve-like support member 104 can include an epaulette capture 450 through which one or more straps 106 connecting the sleeve-like support member 104 and pouch 102 can be placed and secured.

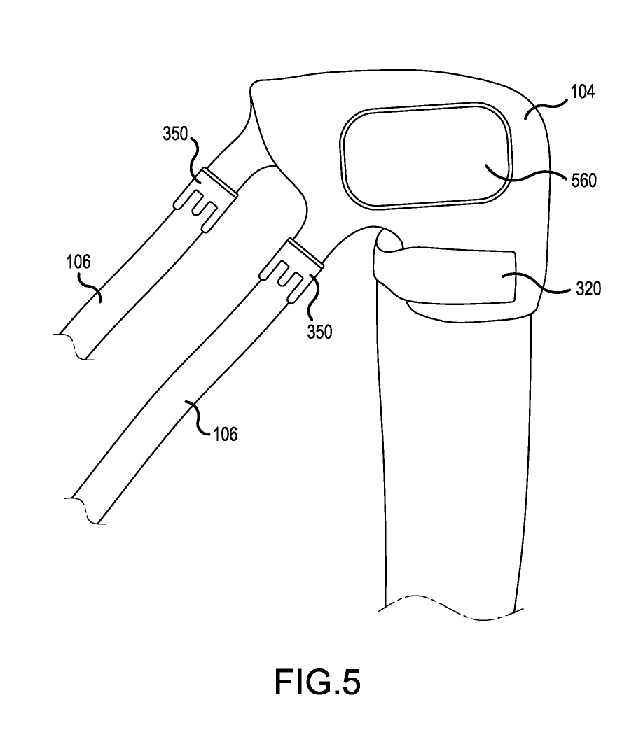

As shown in FIG. 5, the sleeve-like support member 104 can include a generally rectangular aperture 560 in the sleeve-like support member 104 that is adapted to be positioned on the anterior, lateral, or superior aspect of the user's shoulder. The sleeve-like support member 104 can be secured to one or more straps 106 secured to and extending away from a sling pouch (not shown) by buckles 350, hook and loop closures, clasps, or other mechanical fastening means. The sleeve-like support member 104 can be secured to a user's upper arm by an axillary strap 320 positioned near the bottom of the sleeve-like support member 104.

### CONCLUSION

While various preferred embodiments have been shown and described, modifications thereof can be made by one skilled in the art without departing from the spirit and teachings herein. The embodiments herein are exemplary only, and are not limiting. Many variations and modifications of the apparatus disclosed herein are possible and within the scope of the invention. Accordingly, the scope of protection is not limited by the description set out above.

What is claimed is:

1. An apparatus, comprising:
a pouch adapted for receiving a user's arm to be immobilized;
a sleeve-like support member adapted for securing the pouch to the user's shoulder opposite the arm or shoulder to be immobilized;
one or more straps connected to and extending away from the pouch for securing the pouch to sleeve-like support member; and
an axillary strap secured to a bottom portion of the sleeve-like support member and positionable around the user's upper arm.

2. The apparatus of claim 1, further comprising a neck strap connected to a top portion of the sleeve-like support member and adapted to secure the sleeve-like support member around the user's neck.

3. The apparatus of claim 2, further comprising a pad positionable on the neck strap to reduce pressure on the user's neck.

4. The apparatus of claim 1, wherein the sleeve-like support member comprises a generally rectangular aperture.

5. The apparatus of claim 4, wherein the generally rectangular aperture in the sleeve-like support member is adapted to be positioned on the anterior, superior, or lateral aspect of the user's shoulder.

6. The apparatus of claim 1, wherein the sleeve-like support member is detachably connected to the pouch.

7. The apparatus of claim 6, wherein the sleeve-like support member further comprises an epaulette capture for receiving and securing the one or more straps secured to and extending away from the pouch.

8. The apparatus of claim 6, wherein the sleeve-like support member is detachably connected to the pouch by one or more buckles.

9. An apparatus, comprising:
a sleeve-like support member adapted for use with a sling pouch, wherein the sleeve-like support member is shaped to cover a user's shoulder opposite an arm or shoulder of a user to be immobilized.

10. The apparatus of claim 9, further comprising means for securing the sleeve-like support member to a sling pouch.

11. The apparatus of claim 9, wherein the securing means is an epaulette capture, one or more buckles, or a hook and loop closure.

12. The apparatus of claim 9, further comprising an axillary strap for securing the sleeve-liked support member to a user's upper arm.

13. The apparatus of claim 9, further comprising a neck strap connected to a top portion of the sleeve-like support member and adapted to secure the sleeve-like support member around a user's neck.

14. The apparatus of claim 9, further comprising a generally rectangular aperture in the sleeve-like support member adapted to be positioned on the anterior or lateral aspect of a user's shoulder.
